(19) United States
(12) Patent Application Publication
(10) Pub. No.: US 2007/0299133 A1
(43) Pub. Date: Dec. 27, 2007

(54) Concentrated omega-3 fatty acids and mixtures containing them

(76) Inventors: Haile Mehansho, Hamilton, OH (US); Ashok Premchand Luhadiya, Cincinnati, OH (US); Larry Eugene Miller, Cincinnati, OH (US); Amy Violet Trejo, Oregonia, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/821,201

(22) Filed: Jun. 22, 2007

Related U.S. Application Data
(60) Provisional application No. 60/815,992, filed on Jun. 23, 2006.

(51) Int. Cl. A61K 31/22; A23D 7/06

(52) U.S. Cl. 514/547; 426/604

(57) ABSTRACT

A stable aqueous emulsion comprising water, a blend of esters including esters of polyunsaturated fatty acids, such as omega-3 fatty acids, emulsifiers and stabilizers is disclosed. The emulsion may be used as a beverage or as an additive that may be added to a beverage or a food product. The emulsion delivers stable and available omega-3 fatty acids without the undesirable rancid odor.

### CROSS REFERENCED TO RELATED APPLICATIONS

This application claims the benefit of U.S. Provisional Application No. 60/815,992, filed Jun. 23, 2006.

### FIELD OF THE INVENTION

This disclosure relates to triglyceride compositions, such as aqueous emulsions, made from fish oil, vegetable oil or any other oil containing omega-3 fatty acids. The present triglyceride compositions are rich in omega-3 fatty acids and are light colored or colorless and are substantially free of off flavors and objectionable odors. Beverages, food products, and food additives comprising the triglyceride compositions are also disclosed.

### BACKGROUND OF THE INVENTION

Alpha linolenic acid (C18:3; (9Z,12Z,15Z)-Octadeca-9,12,15-trienoic acid, “ALA”), eicosapentaenoic acid (C20:5; (5Z,8Z,11Z,14Z,17Z)-icosa-5,8,11,14,17-pentaenoic acid, “EPA”), and docosahexaenoic acid (C22:6; (4Z,7Z,10Z,13Z,16Z,19Z)-docosa-4,7,10,13,16,19-hexaenoic acid, “DHA”) are long chain polyunsaturated fatty acids having multiple non-conjugated carbon-carbon double bonds with the first of their double bonds at the third carbon from the methyl terminus of the fatty acid and are often collectively referred to as “omega-3 fatty acids” or simply “omega-3s”. Other common omega-3 fatty acids include, but are not limited to, stearidonic acid (C18:4), eicosatetraenoic acid (C20:4), and docosapentaenoic acid (C22:5).

These omega-3 fatty acids are known to have anti-inflammatory functions (enhancing immune response), are effective in the prevention of and therapy for certain thrombotic maladies, for controlling the content of triglycerides in blood in a living system, and for preventing certain thrombotic disturbances (such as, for example, heart attacks, strokes, and the like). Numerous clinical studies have found that omega-3s may further benefit patients with rheumatoid arthritis, high blood pressure, neurodermatitis, and certain other disorders. In response in part to these clinical results, many international institutions and authorities now recommend that individuals increase their daily consumption of omega-3 fatty acids and other polyunsaturated fatty acids (“PUFAs”).

Edible oils, such as fish oil and vegetable oils, are composed of triglycerides. Triglycerides are esters of glycerol with three long chain carboxylic acids (“fatty acids”). In the omega-3 fish oils and vegetable oils, a portion of the triglycerides in the oil include at least one ester of an omega-3 fatty acid. Typically, omega-3 fatty acids are consumed from two sources, in the daily diet and/or as dietary supplements. The primary source of omega-3 fatty acid in the diet is from fish oil and/or vegetables oils rich in omega-3 fatty acids. However, most people do not consume enough fish and/or vegetables rich in omega-3 fatty acids to achieve the recommended levels of consumption of omega-3s. As such, dietary supplements may be necessary for certain people to achieve the health benefits associated with omega-3 fatty acids.

Dietary supplements, however, may present their own problems. For example, one of the richest natural sources of omega-3 fatty acids is fish oil, but only a portion of the triglycerides in natural fish oil contain omega-3 fatty acid esters. Thus, supplements containing fish oil will also contain oils of little health benefit value, yet are high in fat and calories. Moreover, fish oil supplements are typically large gelatinized pills that consumers may find difficult to swallow. In addition, once the dietary supplement has dissolved in the stomach, the fish oil may have a negative affect on a person's breath. Further, fish oil is generally not consumed alone or added to foods or beverages in part because of its pungent odor and fishy flavor.

Fish oils contain varying amounts of omega-3 fatty acids, depending several factors including the type of fish. For example, salmon oil may contain EPA at up to 18% by weight of total fatty acid (“TFA”), and DHA at 12% by weight of TFA. In general, however, the concentration of desirable omega-3 fatty acids is low in fish oils and the amount of fish oil consumed by an average person through normal diet is typically low. While there are natural limits to highly concentrated PUFA triglycerides from fish oils, on account of the triglyceride composition, the typical total content of EPA and DHA in fish oils is approximately 10-25% by weight of TFA.

The fish oils containing omega-3 fatty acids can be obtained as by-products in the production of products such as low-fat fish meal and fish cakes and from oil expression by methods such as boiling or expressing methods. Omega-3 containing fish oil may be obtained from a variety of fish, such as, but not limited to, sardine and/or pilchard, chub mackerel, pacific saury, Alaskan pollack, cod, anchovies, herring, salmon, tuna, and the like. The oil-expressing method employed in obtaining fish oils may be crude and commonly invites lowering of freshness of the material before oil-expression and formation of low-molecular, volatile amines, which are unpleasant smelling substances (e.g. trimethylamine, dimethylamine, and ammonia). Trimethylamine (“TMA”) is one of the major volatile amine compounds associated with the typical “fishy” odor. It is produced by an enzymatic conversion of trimethylamine oxide (“TMAO”), which is an osmo-regulatory compound in many marine fish. During the extraction and storage, the generation and mingling of these unpleasant smelling volatile amines in the fish oil cannot be avoided.

Fish oil also contains amounts of smaller chain length fatty acids, and other highly unsaturated fatty acids in addition to the omega-3s. The double bonds in the fatty acid chains of the omega-3s and other PUFAs in fish oils are susceptible to oxidation by oxygen and other oxidizing agents. The spoilage of fish oil by oxidation and/or bacterial action during storage may result in low molecular weight acids and low molecular weight compounds, such as ketones and aldehydes, in the oil producing undesirable colors, flavors, and/or odors in the oil. Therefore, even though fish oil freshly expressed from natural materials may have no perceptible odor, the production of low molecular amines (TMA) and oxidation products, such as ketones and aldehydes, during storage may give the oil an undesired color, flavor, and/or odor, thereby lowering of the commercial value of the fish oil.

In order to prevent emission of such fish-oil-odors, conventional methods may subject fish oil to refining treatments such as deacidification, deodorization and the like to remove impurities. However, even though conventional refining methods may remove some of the odor causing compound, it may still be impossible to remove completely the volatile amines, aldehydes and ketones since these compounds result from further degradation of the oil or components within the oil upon storage. In addition, there is a tendency in refined or concentrated oils for the emission of fishy odors to become more significant, since the refined fish oil contains higher concentrations of highly unsaturated fatty acids, such as EPA and DHA. Further, trimethylamine and other volatile amine compounds have very low odor threshold values (i.e., very low concentrations of TMA and other volatile amines are readily detected by the human sense of smell). When such oils produced by various commercial methods are incorporated into beverages or food products, the beverages or food products have a noticeable fishy taste and/or smell which many consumers find undesirable.

As such, there exists a need for an emulsion, beverage, food products, or food additive that contains omega-3 fatty acids and has no perceptible fish smell or taste. Moreover, there exists a need for a method of processing commercially available and relatively inexpensive sources of crude fish and vegetable to produce an enriched and concentrated stream of omega-3 fatty acids that can be used in these beverages, food products, and food additives. These and other advantages over prior compositions and processes are discussed in detail in the present disclosure.

### SUMMARY OF THE INVENTION

Embodiments of the present disclosure generally relate to aqueous emulsions comprising esters of polyunsaturated fatty acids, compositions comprising the aqueous emulsions, and processes for forming the emulsions.

In one embodiment, the present disclosure provides a stable aqueous emulsion comprising water, a blend of esters comprising esters of polyunsaturated fatty acids, and one or more compounds capable of reducing a redox potential of the aqueous emulsion. Beverages, food products and food additives comprising the aqueous emulsion are also disclosed.

In another embodiment, the present disclosure provides a method of reducing oxidation in an aqueous emulsion comprising adding a compound to the aqueous emulsion, wherein the aqueous emulsion comprises water and a blend of esters comprising esters of polyunsaturated fatty acids. The compound is a compound capable of reducing the redox potential of the aqueous emulsion.

In another embodiment, the present disclosure provides a method of improving the stability of an aqueous emulsion comprising omega-3 fatty acid esters. The method comprises reducing the redox potential of the aqueous emulsion to less than 600 mV.

Other embodiments of the present disclosure are described in detail in the following specification and claims.

### DETAILED DESCRIPTION OF THE INVENTION

A. Definitions

As used herein, the term “comprising” means various components conjointly employed in the preparation of the compositions of the present disclosure. Accordingly, the terms “consisting essentially of” and “consisting of” are embodied in the term “comprising”.

As used herein, the term “emulsion” means a stable mixture of two immiscible substances in which one substance, the dispersed phase, is dispersed as tiny droplets within the other substance, the continuous phase.

As used herein, the term “fish oil” means a triglyceride oil derived from a fish or other marine organism and comprising triglycerides containing at least one ester of an omega-3 fatty acid.

As used herein, the term “omega-3 fatty acid” means a long chain polyunsaturated fatty acid having a carbon-carbon double bond between the third and fourth carbon from the methyl terminus of the fatty acid chain (i.e., between the omega minus 3 carbon to the omega minus 4 carbon).

As used herein, the term “emulsifier” means a compound or compounds that aids in the formation of an emulsion.

As used herein, the term “stabilizer” means a compound or compounds that help stabilize an emulsion by preventing coalescence of the dispersed phase, such as, for example, by Pickering stabilization or other stabilization process.

As used herein, the term “triglyceride” means a tri-ester formed from glycerol and three fatty acids. The fatty acids may be the same or different. As used herein, the term “diglyceride” means a di-ester of glycerol and two fatty acids, which may be the same or different. The esters may be formed as a 1,2-diester on the glycerol or a 1,3-diester on the glycerol. As used herein, the term “monoglyceride” means a mono-ester of glycerol (either at the 1-hydroxyl group or the 2-hydroxyl group) and a fatty acid.

As used herein, the term “redox potential”, “reduction potential”, or “oxidation-reduction potential” may be used interchangeably and refer to a measurement of the tendency of a compound to gain electrons and thereby be reduced. Reducing the redox potential of a mixture will reduce the likelihood of a compound with a redox potential higher than that of the mixture from becoming oxidized.

As used herein, the term “redox modulator” means a compound or compounds that may be added to a mixture to change the redox potential of the mixture, for example, increasing the redox potential or decreasing the redox potential of the mixture.

As used herein, the term “antioxidant” means a compound or compounds that slow or prevent the oxidation of another chemical species, such as an ester of a polyunsaturated fatty acid. As used herein, the terms “hydrophilic” or “water soluble” when used in reference to an antioxidant means a compound that is highly soluble in water. As used herein, the terms “hydrophobic”, “lipophilic”, or “oil soluble”, when used in reference to an antioxidant means a compound that is relatively insoluble in water and soluble in a lipid or oil.

As used herein the term “chelator” means a compound or ligand that binds or chelates to a metal or metal ion species to form a metal complex or chelate. The metal complex or chelate may be less reactive than the non-chelated metal species.

As used herein, the terms “per serving”, “per unit serving”, or “serving size” when used in the context of a liquid, such as the emulsions, or beverages herein, refers to a volume of the final emulsion or beverage formulation in milliliters.

The U.S. Recommended Daily Intake (“USRDI”) for vitamins and minerals are defined and set forth in the Recommended Daily Dietary Allowance-Food and Nutrition Board, National Academy of Sciences National Research Council, for a serving size of 250 mL of an aqueous composition.

As used herein, all parts, percentages, and ratios are based on weight unless otherwise specified.

Emulsions

According to various embodiments, the present disclosure provides an aqueous emulsion comprising water, an emulsifier, a stabilizer, and a blend of esters comprising esters of polyunsaturated fatty acids. In certain embodiments, the blend of esters may comprise a concentrate, such as concentrated blend described herein, for example, a blend of esters comprising greater than 50% of esters of fatty acids having 20 carbons or more. According to certain embodiments, the emulsions may comprise a beverage or may be added to a beverage. In other embodiments, the emulsions may be added to a food product. In still other embodiments, the emulsion may be a pre-mixed food additive that may be provided to an end user for addition to a food product or beverage. Addition of the emulsion to a food product or beverage may be used to increase the content of the polyunsaturated fatty acid in the food product or beverage.

Esters of polyunsaturated fatty acids that may be used in various embodiments of the present disclosure include, esters of fatty acids having from 18 to 26 carbon atoms with from 2 to 6 carbon-carbon double bonds. In other embodiments, the esters of fatty acids may have from 20 to 22 carbon atoms with from 2 to 6 carbon-carbon double bonds. The carbon-carbon double bonds will typically have the “cis” or “Z” configuration and in various embodiments, the double bonds will be non-conjugated (i.e., separated by more than one carbon-carbon single bond). In certain specific embodiments, the esters of the fatty acids may be esters of omega-3 fatty acids, such as, but not limited to ALA, EPA, and DHA).

The esters may be in the form of a blend of esters, such as, for example, a fish oil or vegetable oil comprising triglycerides high in polyunsaturated fatty acids, for example omega-3 fatty acids. The fish oil or vegetable oil may be a crude oil, a partially refined oil, a refined oil, or an oil concentrate. Suitable fish oils include any marine oil that contains polyunsaturated fatty acids, such as, but not limited to oils derived from sardine, pilchard, chub mackerel, pacific saury, trout, pollack, cod, anchovies, herring, salmon, tuna, and the like. Suitable vegetable oils may include flax or linseed oil, hemp oil, soya oil, canola (rapeseed) oil, chia seed oil, pumpkin seed oil, perilla seed oil, purslane, sunflower seed oil, and nuts (walnut, pistachio, peanut, almond, etc., and their respective oils).

The esters of fatty acids will comprise esters of polyunsaturated fatty acids, such as omega-3 fatty acids. According to certain embodiments, the esters of the polyunsaturated fatty acids may include C₁-C₄ alkyl esters, including ethyl esters and propyl esters, propylene glycol monoesters (i.e., monoesters of 1,2-propanediol), propylene glycol diesters, monoglycerides, diglycerides, triglycerides and combinations of any thereof. Esters comprising diglycerides may be 1,2-diglycerides or 1,3-diglycerides and esters comprising monoglycerides may be 1-monoglycerides or 2-monoglycerides. In esters comprising propylene glycol monoesters, the ester functionality may be attached at either the 1-hydroxy group or the 2-hydroxy group. In those embodiments where ester is a diester or a triester, such as a propylene glycol diester, a diglyceride, or a triglyceride, at least one of the ester groups is an ester of a polyunsaturated fatty acid, such as an omega-3 fatty acid. That is, one, two, or, in the case of triglycerides, all three of the esters may be ester functional groups of a polyunsaturated fatty acid. However, the other ester(s) of the diester or triester may be a mono- or unsaturated fatty acid. Further, the fatty acids forming the esters in the diesters and triesters may be the same or different. In specific embodiments, the esters are triglycerides comprising omega-3 fatty acids, wherein at least one of the ester groups on the triglyceride is an ester of an omega-3 fatty acid.

According to certain embodiments, the blend of esters comprising esters of polyunsaturated fatty acids may be a concentrated blend of esters of polyunsaturated fatty acids. Concentrated blends of esters are blends of esters where the content of the polyunsaturated fatty acid (such as omega-3 fatty acid) has been increased by some refining or treatment process. Concentrated blends of esters of polyunsaturated fatty acids may be made by any process known in the art. In specific embodiments, the concentrated blend of esters may be made by the process as described herein.

The ratio of the different omega-3 fatty acids, such as ALA, EPA, and/or DHA, in the esters of polyunsaturated fatty acids may vary according to the source of the esters or the process through which they are produced. For example, different species of fish may have fish oil with differing amounts of the omega-3 fatty acids. In addition, the diet of the fish or the season of the year may also affect the amount and type of omega-3 fatty acid residues in the fish oil. Vegetable oil sources of omega-3 fatty acids may have different types and ratios of the omega-3 fatty acids compared to marine sources. For blends of esters of omega-3 fatty acids that are produced or refined by a chemical or physical process, the type and ratio of fatty acid residue may also vary. For certain processes, the type and ratio of omega-3 fatty acids in the blend may be controlled to give a desired ratio. For example, in certain embodiments a blend that is high in DHA residues may be desired and thus the production process may be controlled to provide a blend high in DHA. In other embodiments, a blend with an approximately equal ratio of EPA and DHA or blends with high concentrations of EPA may be produced. According to one embodiment, the ratio of EPA to DHA may be 1.5:1, for example in a blend of esters which comprises about 18% of EPA and 12% DHA. The emulsion compositions of the present disclosure should not be limited by the ratio of fatty acid residues in the blend of esters, since different blends of omega-3 esters of fatty acids are contemplated. Specifically, the ratio of the blends of omega-3 fatty acids in the emulsions may depend on the intended final use.

The concentration of the polyunsaturated fatty acids in the emulsion may be varied according to the amount of the blend of esters dispersed in the water and the concentration of the polyunsaturated fatty acids in the blend of the esters. The concentration of the esters of polyunsaturated fatty acid may be measured, for example, as a weight of fatty acids residues per serving size or weight of the emulsion. For example, in certain embodiments, the blend of esters may comprise a blend of esters of omega-3 fatty acids. According to certain embodiments, the concentration of omega-3 fatty acid in the emulsion may be from about 100 mg to about 5 g of omega-3 fatty acids per 100 mL of the emulsion. In other embodiments, the concentration of omega-3 fatty acid in the emulsion may be from about 135 mg to about 4 g. The emulsions may comprise differing amounts of the specific omega-3 fatty acid residues. For example, in certain embodiments, the emulsions may comprise from 0 mg to about 2 g of DHA and from 0 mg to about 2 g of EPA per serving. In certain embodiments, the emulsions may comprise at about 150 mg to about 2 g of DHA and about 150 mg to about 2 g of EPA per serving. In other embodiments, the emulsions may comprise about 250 mg to about 2 g of DHA and about 250 mg to about 2 g of EPA in each serving and in still other embodiments, about 350 mg to about 2 g of DHA and about 350 mg to about 2 g of EPA in each serving. The concentration of omega-3 fatty acid in the emulsion may be varied to deliver a desired amount of the omega-3 fatty acid, for example, an amount equivalent to a recommended daily consumption.

In certain embodiments the emulsion may comprise a pH of less than 4.5, such as a pH within a range as disclosed herein. In other embodiments, the emulsion may comprise a compound capable of reducing the redox potential of the aqueous emulsion. Examples of such compounds are set forth in greater detail herein. In specific embodiments, the aqueous emulsion may comprise at least one of a pH of less than 4.5 and a compound capable of reducing the redox potential of the aqueous emulsion.

According to other embodiments, the aqueous emulsions of the present disclosure may further comprise at least one additive selected from the group consisting of an artificial sweetener, a natural sweetener, an artificial flavor, a natural flavor, a redox modulator, an edible acid or acidulant, a preservative, a colorant, and combinations of any thereof. Various suitable additives are known in the art or are disclosed in greater detail herein.

Other embodiments of the aqueous emulsions may be in the form of a dry powder, which may be produced by drying the emulsion by a suitable method, such as, for example, spray drying, spray congealing, dry blending with carriers such as cyclodextrin, etc. The dried emulsion powder may then be reconstituted by adding water to the powder and mixing to rehydrate the powder and reconstitute the emulsion at the desired levels of omega-3 fatty acids in the finished product for intended consumption.

Emulsifiers and Stabilizers

The aqueous emulsion according to the various embodiments disclosed herein may comprise an emulsifier and a stabilizer. The emulsifier may be added to the emulsion to assist in the formation of the emulsion, for example by reducing the surface tension on the surface of the droplets of the lipophilic esters and assisting in the dispersion of the dispersed phase as small droplets in the continuous phase. Stabilizers may assist in stabilizing the emulsion by preventing coalescence, flocculation, or creaming of the dispersed phase, such as by creating a repulsive interaction between the droplets of the dispersed phase, for example, by creating an ionic charge on the droplet surface which is repelled by the like charge on the surfaces of the other droplets.

According to certain embodiments, suitable emulsifiers may include, but are not limited to lecithins, cephalins, plasmalogens, phosphatidyl choline, phosphatidylethanolamine, phosphatidylinositol, cerbroside, sorbitan esters of long chain saturated fatty acids, lactic acid esters of long chain saturated fatty acid monoglycerides, diacetyl tartaric acid esters of long chain saturated fatty acid monoglycerides, monoglycerides, diglycerides, stearoyl lactate, bile salts, bile acids, and combinations of any thereof. In specific embodiments, the emulsifier may be lecithin.

According to certain embodiments, suitable stabilizers may include proteins, sterols, and gums such as, but are not limited to whey proteins, caseins, soy proteins, animal and plant sterols, sucrose esters of long chain fatty acids, agar, carrageenan, xanthan gum, pectin, guar gum, gum Arabic, gellan gum, sodium carboxymethylcellulose, hydroxypropyl cellulose, locust bean gum, animal and plant sterols (such as, for example, cholesterol, stigmasterol), polyphenols (e.g. green tea extracts), sucrose esters of long chain fatty acids and combinations of any thereof. In specific embodiments, the stabilizer may a whey protein.

According to other embodiments, the present disclosure may provide an emulsifier-stabilizer composition for forming a stable aqueous emulsion of a triglyceride oil. The triglyceride oil may be a vegetable oil or a fish oil comprising saturated fatty acids, monounsaturated fatty acids, polyunsaturated fatty acids, and combinations of any thereof. In specific embodiments, the triglyceride oil may comprise at least one ester group that is an omega-3 fatty acid ester. In other embodiments, the triglyceride oil is a marine oil, such as a fish oil. The emulsifier-stabilizer composition may comprise a phospholipid emulsifier and a protein-based stabilizer.

According to various embodiments, the phospholipid emulsifier may be selected from the group consisting of lecithins, cephalins, plasmalogens, phosphatidyl choline, phosphatidylethanolamine, phosphatidylinositol, and combinations of any thereof. In specific embodiments, the phospholipid emulsifier is lecithin. The phospholipid emulsifier may be added to the aqueous emulsion in an amount sufficient to make a triglyceride oil to phospholipid emulsifier ratio ranging from about 20 to about 85 parts triglyceride oil to 1 part phospholipid emulsifier.

According to certain embodiments, the protein-based stabilizer may be selected from the group consisting of a whey protein, a casein, a soy protein, hydrolyzates and partial hydrolyzates thereof, and combinations of any thereof. In specific embodiments, the protein-based stabilizer is a whey protein. The protein-based stabilizer may be added to the aqueous emulsion in an amount sufficient to make a triglyceride oil to protein-based stabilizer ratio ranging from about 1 to about 10 parts triglyceride oil to 1 part protein-based stabilizer.

In one specific embodiment, the phospholipid emulsifier is lecithin and the protein-based stabilizer is a whey protein. According to one embodiment, the lecithin may be added to the emulsion in an amount sufficient to make a triglyceride oil to lecithin ratio ranging from about 20 to about 85 parts triglyceride oil to 1 part lecithin and the whey protein may be added to the emulsion in an amount sufficient to make a triglyceride oil to whey protein ratio ranging from about 1 to about 10 parts triglyceride oil to 1 part whey protein.

Certain embodiments of the present disclosure provide methods of producing a stable aqueous emulsion of a triglyceride oil, such as the triglyceride oils described herein (including triglycerides having at least one omega-3 fatty acid ester). The methods may comprise combining water, the triglyceride oil, and an emulsifier-stabilizer composition to form a mixture, and mixing the mixture to produce the aqueous emulsion. The emulsifier-stabilizer composition may be any of the phospholipid emulsifier and protein-based stabilizer compositions described herein. The phospholipid emulsifier may be added in an amount as described herein and the protein-based stabilizer may be added to the emulsion in an amount as described herein. According to specific embodiments, the phospholipid emulsifier is lecithin. According to other embodiments, the protein-based stabilizer is a whey protein. In one specific embodiment of the method, the emulsifier is lecithin and the stabilizer is a whey protein.

Other specific embodiments of the present disclosure provide methods of producing a stable aqueous emulsion of an ester of an omega-3 fatty acid. The method may comprise combining water, the ester of the omega-3 fatty acid, an emulsifier-stabilizer composition comprising a lecithin emulsifier and a whey protein stabilizer to form a mixture and mixing the mixture to produce the aqueous emulsion. According to certain embodiments of the method, the lecithin may be added in an amount sufficient to make a ester of an omega-3 fatty acid to lecithin ratio ranging from about 20 to about 85 parts ester of an omega-3 fatty acid to 1 part lecithin and the whey protein may be added in an amount sufficient to make a ester of an omega-3 fatty acid to whey protein ratio ranging from about 1 to about 10 parts ester of an omega-3 fatty acid to 1 part whey protein.

Acid Component

An edible acid or food grade acid or acidulant can optionally be added to the aqueous emulsions of the present disclosure. For example, in certain embodiments it may be desirable to regulate the pH of the emulsion. These acids may be used alone or in combination.

In certain embodiments, blends of esters of polyunsaturated fatty acids, such as omega-3 fatty acids, wherein the blend is derived from a marine or fish oil may have an undesirable fishy odor and taste due to the presence of volatile amine compounds. Trimethylamine (“TMA”) is one of the major volatile amine compounds associated with the typical “fishy” odor and flavor of fish oils. TMA is produced by an enzymatic conversion of trimethylamine oxide, which is an osmo-regulatory compound in many marine fish. During the extraction, processing, and storage, the generation and mingling of these unpleasant smelling volatile amines in the fish oil cannot be avoided. When producing an emulsion of esters of polyunsaturated fatty acids derived from fish oil, a fishy odor due to the presence of volatile amine compounds, such as TMA, in the headspace above the emulsion may be perceived by the human sense of smell, even at low TMA concentration, such as concentrations above 50 part per billion (ppb), in other cases at concentrations above 1 ppb, and in certain cases at concentration levels above 210 part per trillion (ppt). The presence of the volatile amines, such as TMA, in the oil may also result in a fishy flavor associated with an emulsion that comprises the oil. For example, a fishy flavor in the emulsion may be perceived at TMA concentrations of more than 50 ppb, in certain embodiments at concentrations of greater than 1 ppb and in other embodiments at concentrations of greater than 210 ppt based on the total volume of emulsion. Thus, certain embodiments of the present disclosure provide for an aqueous emulsion, such as described herein, which has no perceptible fish or fishy odor. That is, the emulsion has reduced concentration of volatile amine compounds, such as TMA, both in the oil in the emulsion and in the head space above the emulsion.

In one embodiment, the present disclosure provides for an aqueous emulsion having no perceptible fish odor. The aqueous emulsion comprises water and an oil comprising a blend of esters of polyunsaturated fatty acids, such as a blend of triglycerides having at least one omega-3 fatty acid wherein the aqueous emulsion has a pH of less than about 4.5. In certain embodiments, the aqueous emulsion may have a pH ranging from about 2 to about 4. In other embodiments, the aqueous emulsion may have a pH ranging from about 2.5 to about 3.7. While certain embodiments may have a lower pH limit of about 2, emulsions having pH's less than 2 are also contemplated and should be considered within the scope of the present disclosure. For example, in embodiments wherein the aqueous emulsions comprise a food additive that may be added to a food product, emulsions of lower pH may be utilized and added in amounts such that the pH of the food product comprising the food additive ranges from about 2 to about 4. However, in certain applications, such as those involving food products or beverages, lower pH's may not be optimum and therefore in those applications emulsions having the recited pH ranges may be desired.

Without intending to be limited by any explanation, it is believed that the volatile amines compounds, such as TMA, which may be present in a blend of triglycerides, such as a fish oil, within the aqueous emulsions of the present disclosure will be protonated under the acidic conditions associated with the lower pH values described herein. That is, at the pH values recited herein, the volatile amine compounds will be in form of an ammonium salt and not the free amine. It is believed that the increased vapor pressure associated with the ionic ammonium salts will reduce the concentration of the free amine in the head space above the emulsion and therefore reduce the perceived fishy odor. For example, free TMA will react with an acidulant under the low pH conditions associated with the emulsion to form an ammonium salt of the (CH₃)₃NH⁺ ion along with the counter ion associated with the conjugate base of the acidulant. In addition, the compositions in which the free volatile amine compounds are converted to their ammonium salts may have less of a fishy flavor.

According to certain embodiments, the aqueous emulsions of the present disclosure may comprise a food grade acidulant such as, but not limited to, malic acid, tartaric acid, citric acid, phosphoric acid, acetic acid, lactic acid, fumaric acid, adipic acid, succinic acid, glycono-delta-lactone, succinic anhydride, carbonic acid, and combinations of any thereof. In one specific embodiment, the aqueous emulsions of the present disclosure may comprise an acidulant that is a mixture of phosphoric acid and malic acid. The acidulant may be added in sufficient amount necessary to reduce the pH of the aqueous emulsion to the desired pH level, as recited herein. In view of the present disclosure, one skilled in the art could readily determine the amount of acidulant necessary to reduce the pH of the emulsion to the desired levels.

As recited herein, the aqueous emulsions of the present disclosure may have no perceptible fish taste or odor, for example, by having a reduced concentration of free or undissolved volatile amine compounds, such as free TMA. In certain embodiments, the aqueous emulsion may have a concentration of free or undissolved TMA of less than the detection limit of human taste or sense of smell. That is, a person consuming the emulsion or a composition comprising the emulsion would not taste or smell the TMA. In specific embodiments, the concentration of free or undissolved TMA in the aqueous emulsion may be less than 50 ppb, in certain embodiments less than 1 ppb, and in still other embodiments less than 210 ppt.

In other embodiments, the head space above the aqueous emulsion or a composition that comprises the aqueous emulsion will have a concentration of volatile amine compounds, such as TMA, that is less than the detection limit of the human nose and sense of smell. The human nose and smell receptors therein are particularly sensitive to volatile amine compounds, including TMA, such that even small concentrations of the amines can be detected. For example, certain volatile amines may be detected even at concentrations as low as about 1 ppb and for certain amines as low as about 210 ppt. For certain amine compounds the detection limit may be as low as about 32 ppt. In various embodiments, the emulsions of the present disclosure have no perceptible fish odor or odor associated with volatile amine compounds. Thus, certain embodiments of the aqueous emulsion or compositions that comprise the aqueous emulsion may have a concentration of volatile amine compounds, such as TMA, in the head space above the composition that is less than about 1 ppb. In certain embodiments, the aqueous emulsion may have a head space concentration of volatile amine compounds, such as TMA, of less than about 210 ppt. In other embodiments, the aqueous emulsion may have a head space concentration of volatile amine compounds, such as TMA, of less than about 32 ppt.

According to certain embodiments, the present disclosure includes methods of reducing an amount of volatile amine compounds, which may comprise TMA, in an aqueous' emulsion, such as the emulsions of the present disclosure. The methods may comprise adding an acidulant to the emulsion in an amount sufficient to decrease the pH of the aqueous emulsion to less than 4.5. In certain embodiments, the acidulant is added in an amount sufficient to decrease the pH to a range of about 2 to about 4 and in other embodiments to a range of about 2.5 to 3.7. In certain embodiments of the methods, the aqueous emulsion may comprise an omega-3 fatty acid ester, which in specific embodiments, comprises a triglyceride having at least one omega-3 fatty acid ester and in other embodiments may comprise a fish oil or a concentrate of omega-3 fatty acids (such as the concentrates described herein). The acidulants according to certain methods may be selected from the group consisting of malic acid, tartaric acid, citric acid, phosphoric acid, acetic acid, lactic acid, fumaric acid, adipic acid, succinic acid, glycono-delta-lactone, succinic anhydride, carbonic acid, and combinations of any thereof. In certain embodiments, the methods comprise reducing the concentration of volatile amine compound in the head space above the emulsion or a composition comprising the emulsion to a value less than the detection limit of the human nose, such as the volatile amine concentrations described herein.

Other embodiments of the method may further comprise adding the aqueous emulsion to a beverage or a food product. For example, as described in greater detail elsewhere herein, the method may comprise using the aqueous emulsion as a pre-mixed product that may be added to a beverage or food product to increase the amount polyunsaturated fatty acids, such as omega-3 fatty acids, in the beverage or food product.

Redox Modulators

According to certain embodiments, the present disclosure provides for an aqueous emulsion comprising water, a blend of esters comprising esters of polyunsaturated fatty acids, and one or more compounds capable of reducing a redox potential of the aqueous emulsion. The esters of polyunsaturated fatty acids may be esters of omega-3 fatty acids, such as triglycerides having at least one omega-3 fatty acid ester, for example, a fish oil or a concentrate of omega-3 fatty acid esters. The carbon-carbon double bonds of the polyunsaturated fatty acids in the esters of the emulsions may be susceptible to oxidation. For example, oxidation of lipids by oxygen, either in the form of triplet oxygen or singlet oxygen, may result in decomposition products of the polyunsaturated fatty acid, such as aldehydes and ketones, which can result in undesired off-flavors, colors, or odors in the emulsion. For example, singlet oxygen may react directly with a double bond in the fatty acid, or triplet oxygen may react with an activated radical of a fatty acid. In either case, peroxides may be formed which may then decompose to aldehydes, ketones, and/or other byproducts. By reducing or inhibiting oxidation of the double bonds in the fatty acids, the resulting emulsion and compositions comprising the emulsion will not have the off-flavors, colors, and odors associated with lipid oxidation and the resulting compositions will demonstrate a higher stability, for example, a longer shelf life.

Polyunsaturated fatty acids and their esters, such as omega-3 fatty acids, have a redox potential of about 600 mV. That is, the carbon-carbon double bonds in the polyunsaturated fatty acid chain may be oxidized by a compound having a higher redox potential than 600 mV. In certain embodiments, a factor in maintaining the stability of the omega-3 fatty acids in the aqueous formulations of the present disclosure is the control of the redox potential of the emulsions or compositions comprising the emulsions. In the presence of water, various food components and compounds may undergo oxidation-reduction reactions, in an equilibrium state that is dictated by the redox potential of the aqueous system. In the case of omega-3 fatty acids, a redox potential below 600 mV will favor less oxidation of the carbon-carbon double bonds in the fatty acid residue. Thus, in one embodiment, the redox potential of the aqueous emulsions or compositions comprising the emulsions is maintained below about 600 mV. In other embodiments, the redox potential may be maintained below about 500 mV, in other embodiments below 400 mV, in still other embodiments below about 300 mV, in still other embodiments below about 200 mV, and in specific embodiments below about 150 mV.

One approach to limit oxidation of the fatty acid residues in the emulsions of the present disclosure may be to reduce the redox potential of the aqueous emulsion by the addition of one or more compounds having a redox potential less than the polyunsaturated fatty acid. The one or more compounds may be added in an amount sufficient to reduce the redox potential of the compositions to a value such as those described herein. In this approach, the one or more compound having the lower redox potential may stabilize the omega-3 fatty acids by scavenging/reacting with the oxidizing agent before the polyunsaturated fatty acids, and changing the environment from oxidizing into reducing. Such omega-3 fatty acid stabilizing aqueous environment may be defined by the following equation:
0>RP−(A−B*pH)

Wherein RP, pH are the redox potential (in mV) and pH of the aqueous system. In this equation A is 400 or less and B is 16.

According to various embodiments, the one or more compounds capable of reducing the redox potential of the aqueous emulsion may include, but are not limited to, compounds selected from the group consisting of a compound that lowers the pH of the aqueous emulsion, a redox modulator, a reducing agent, a chelator, an antioxidant, and combinations of any thereof. Compounds that lower the pH of the aqueous emulsion are described in greater detail with regards to acidulants.

One approach to lower the redox potential of the aqueous emulsion comprises adding one or more compounds that lowers the pH of the aqueous emulsion, such as, by adding an acidulant as described herein. In addition to reducing the amount of free or dissolved volatile amine in the composition, adding one or more compounds capable of lowering the pH of the aqueous emulsion will also lower the redox potential of the emulsion thereby reducing the oxidation of the polyunsaturated fatty acid residues within the emulsion. The one or more compounds may be added in an amount sufficient to reduce the pH of the emulsion to the desired levels, such as a pH less than about 4.5, or other level as described herein.

In certain embodiments, the one or more compounds capable of reducing the redox potential of the aqueous emulsion is a redox modulator. Redox modulators include compounds that have the property of converting the oxidizing environment of regular water to reducing environment. Normally, the redox modulators have electron donating functional groups. These electron donating compounds keep minerals in a reduced and soluble form, and prevent vitamins and flavors from degradation by reducing the redox potential of the vehicle water. Suitable redox modulators may include, but are not limited to, ascorbic acid, ascorbyl palmitate, sodium bisulfite, erythorbic acid, sulfhydryl containing amino acid residues (i.e., amino acids, peptides, and proteins containing the thiol functional group, for example cysteine), polyphenols, flavonoids, soluble dietary fibers (e.g. arabinogalactan), and combinations of any thereof. In specific embodiments, the redox modulator may be one of ascorbic acid, erythorbic acid, and sodium bisulfite. The redox modulator may be added in an amount sufficient to reduce the redox potential of the aqueous emulsion to less than 600 mV. In certain embodiments, the redox modulator is added in an amount sufficient to reduce the redox potential to less than 500 mV and in specific embodiments, to less than 400 mV or the other redox potentials as set forth herein.

According to other embodiments of the emulsions, the one or more compounds capable of reducing the redox potential of the aqueous emulsion may be a chelator, such as, compounds with two or more electron donating groups, including, but not limited to, ethylenediamine tetraacetic acid (“EDTA”), citric acid, tartaric acid, ascorbic acid, polyphosphates, esters or salts thereof, and combinations of any thereof. Chelators are compounds that bind to metal ions to form a metal/chelate complex. Metal ions, such as certain transition metal ions, may act as oxidizing agents and may oxidize a carbon-carbon double bond in the polyunsaturated fatty acid by abstraction of an H. (i.e., a hydrogen radical) from a methylene alpha to the double bond, resulting in an allylic radical, which may then react with triplet oxygen to form a peroxy radical. The peroxy radical may then abstract a H. (hydrogen radical) from another fatty acid chain during a propagation step. When a chelator chelates to a metal ion in the composition, the metal ion may no longer be able to react with the fatty acid chain, thereby inhibiting the oxidation process. The chelators described herein may be suitable to chelate to a number of transition metal ions, such as, for example, Fe²⁺, Cu²⁺, Mn²⁺, Co²⁺, Fe³⁺, Mn²⁺, Ni²⁺, and mixtures of any thereof. Other chelators known to one skilled in the art that may react with these and other metal ions may also be used in certain embodiments of the emulsions.

In still other embodiments, the one or more compounds capable of reducing the redox potential of the aqueous emulsion may be a combination of a hydrophilic or water soluble antioxidant and a hydrophobic or oil soluble antioxidant. According to these embodiments, the water soluble antioxidant may be dissolved in the aqueous portion (continuous phase) of the aqueous emulsion whereas the oil soluble antioxidant may be soluble in the dispersed phase of the emulsion (i.e., the oil droplets of the blend of esters comprising esters of polyunsaturated fatty acids). Antioxidants typically inhibit oxidation of an oxidation susceptible compound, such as the unsaturated fatty acids, by reacting preferentially with the oxidizing agent before the oxidizing agent reacts with the compound. The product of the reaction between the antioxidant and the oxidizing agent is typically inert or non-reactive and should also be tasteless, colorless, and odorless so as not to affect the taste, smell, or look of the product. By utilizing a combination of a water soluble antioxidant and an oil soluble antioxidant in the emulsion, oxidants in either the continuous phase (i.e., the water phase) and the dispersed phase (i.e., the blend of esters) may be neutralized and prevented from reacting with the fatty acid carbon-carbon double bonds. Water soluble antioxidants suitable for use in the various embodiments of the aqueous emulsions include, but are not limited to, ascorbic acid, erythorbic acid, a botanical extract, such as rosemary extract, green tea extract, or other extract containing a polyphenol antioxidant, and combinations thereof. Oil soluble antioxidants suitable for use in the various embodiments of the aqueous emulsion include, but are not limited to, vitamin E, tocopherols, ascorbyl palmitate, butylated hydroxyanixole (“BHA”), butylated hydroxytoluene (“BHT”), and combinations of any thereof. The water soluble antioxidant and the oil soluble antioxidant may be added to the emulsion in an amount sufficient to inhibit the oxidation of the fatty acid residues. For example, the antioxidants may be added in sufficient amount to reduce the redox potential of the emulsion, such as to the levels recited herein.

According to other embodiments, the present disclosure also provides methods of reducing oxidation of esters of polyunsaturated fatty acids, such as omega-3 fatty acid esters, in an emulsion. The methods comprise adding a compound to an aqueous emulsion comprising water and a blend of esters comprising esters of polyunsaturated fatty acids, wherein the compound is capable of reducing the redox potential of the aqueous emulsion. According to specific embodiments, the esters of polyunsaturated fatty acids comprise esters of omega-3 fatty acids, such as triglycerides having at least one omega-3 fatty acid ester. Examples of such blends of esters include edible oils containing polyunsaturated fatty acid esters, such as fish oils and certain vegetable oils, as set forth in greater detail herein. The compound may be selected from the group consisting of a compound that lowers the pH of the aqueous emulsion, a redox modulator, a reducing agent, a chelator, an antioxidant, and combinations of any thereof. Examples of such compounds are set forth in greater detail herein.

According to other embodiments, the present disclosure provides methods of improving the stability of an aqueous emulsion comprising omega-3 fatty acid esters. The methods comprise adding a compound to the aqueous emulsion, wherein the compound is capable of reducing the redox potential of the aqueous emulsions. Still other methods of improving the stability of the aqueous emulsion may comprise reducing the redox potential of the aqueous emulsion to a value less than the redox potential of the omega-3 fatty acid esters, such as, less than 600 mV, in certain embodiments less than 500 mV, and in other embodiments less than 400 mV. The omega-3 fatty acid esters may be triglycerides comprising at least one ester of an omega-3 fatty acid, including those natural and processed oils set discussed herein. The compound may be selected from the group consisting of a compound that lowers the pH of the aqueous emulsion, a redox modulator, a reducing agent, a chelator, an antioxidant, and combinations of any thereof. Examples of such compounds are set forth in greater detail herein.

Applications

In certain embodiments, the aqueous emulsion according to any of the various embodiments described herein may be incorporated into a beverage or a food product. For example, according to one embodiment, the present disclosure contemplates a beverage comprising any of the aqueous emulsion described herein. Specific beverages are discussed in greater detail herein. In other embodiments, the present disclosure contemplates a food product comprising any of the stable aqueous emulsions described herein. Beverages and food products comprising the stable aqueous emulsions described herein may be used, for example, to increase the content of polyunsaturated fatty acids, such as omega-3 fatty acids, in a diet. The beverages and food products described herein allow for the ready consumption of polyunsaturated fatty acids and allow a consumer to obtain the benefits associated with a diet high in polyunsaturated fatty acids (such as the omega-3 fatty acids). Other embodiments allow for the formation of beverages and food products that contain emulsions wherein the emulsion comprises a blend of omega-3 fatty acid ester derived from a marine or fish oil or a concentrate of omega-3 fatty acid esters (such as described herein). As discussed in detail herein, such oils typically have an unpleasant fishy odor and flavor due to the presence of volatile amine compounds, such as TMA, and other degradation products from oxidation processes on the fatty acids. However, as set forth herein, such beverages and food products comprising the aqueous emulsions comprising fish oil derived omega-3 fatty acids produced according to the methods recited herein will not have these unpleasant odors and flavors due to the composition and method of production. Such aqueous emulsions and beverages and food products formed therefrom will be commercially desirable since many consumers prefer beverages and food products without the off odors and flavors generally associated with fish oils.

Other embodiments of the present disclosure provide for a food additive comprising any of the stable aqueous emulsions described herein. A food additive comprising the aqueous emulsion may include a pre-mixed emulsion that may be provided to an end user food producer who, for example, wants to produce a product that contains increased levels of polyunsaturated fatty acid, such as omega-3 fatty acid, but does not want the product to have undesired odors and/or flavors, such as those associated with free or undissolved volatile amine compounds and/or lipid oxidation products. The food additives according to these embodiments may include esters of polyunsaturated fatty acids that are triglycerides having at least one ester group on the triglyceride being an ester of an omega-3 fatty acid, such as a fish or marine oil, a vegetable oil having omega-3 fatty acid esters, or a concentrated oil produced by a process such as those described herein or other known process.

Thus, according to certain embodiments, the present disclosure provides methods for increasing the content of polyunsaturated fatty acids, such as, for example, omega-3 fatty acids, in a food product or beverage. The methods may comprise adding a stable aqueous emulsion to the food product or beverage, wherein the stable aqueous emulsion comprises water, an emulsifier, a stabilizer, and a blend of esters comprising esters of polyunsaturated fatty acids, such as omega-3 fatty acids. In certain embodiments, the polyunsaturated fatty acid esters may be esters of triglycerides wherein at least one of the ester groups of the triglyceride is an ester of an omega-3 fatty acid, including for example, the concentrate produced by the process disclosed herein. In specific embodiments, the aqueous emulsion may further comprise a lecithin emulsifier and a whey protein stabilizer. According to specific embodiments, the aqueous emulsion of the food additive may comprise a redox modulator as set forth in detail herein.

In specific embodiments, particularly those comprising polyunsaturated fatty acid esters derived from fish oil, the food additive may have no perceptible fish odor or flavor. According to these embodiments, the present disclosure provides a food additive comprising the stable aqueous emulsion comprising triglycerides having at least one polyunsaturated fatty acid, such as an omega-3 fatty acid, wherein the food additive has a pH of less than about 4.5 and has no perceptible fishy odor or flavor. Other embodiments of the food additive may have a pH ranging from about 2 to about 4 and in certain embodiments from about 2.5 to about 3.7. In a specific embodiment, the food additive may have a head space above the food additive with a concentration of volatile amine compounds, such as TMA, that is less than the detection limit of the human nose. In other embodiments the volatile amine compound concentration may be less than those limits associated with the aqueous emulsion.

Other embodiments of the stabilized emulsion comprising polyunsaturated fatty acid esters, such as omega-3 fatty acid esters, may further comprise one or more compounds capable of reducing the redox potential of the emulsion. As discussed in detail herein, emulsions comprising the one or more compound will have an environment in which oxidation of the carbon-carbon double bonds of the polyunsaturated fatty acids is inhibited. Thus, the emulsions and compositions that comprise the emulsions will have lower concentrations of polyunsaturated fatty acid oxidation products, such as certain ketones and aldehydes, and will lack the off flavors and smells associated with a composition comprising the products for oxidized fatty acids.

Other embodiments of the present disclosure provide a method of providing a food additive having a high concentration of polyunsaturated fatty acids, including omega-3 fatty acids, to a food producer. The method comprises mixing water, an emulsifier, a stabilizer, and a blend of esters comprising esters of polyunsaturated fatty acids (such as omega-3 fatty acids, including triglycerides wherein at least one ester is an ester of an omega-3 fatty acid) to form a stable aqueous emulsion and providing the stable aqueous emulsion to a food producer. The various embodiments of the stable aqueous emulsion as described by the compositions and methods herein are suitable to be used in these methods. According to certain embodiments, the method may further comprise processing and packaging the food additive.

Beverages

In certain embodiments, the present disclosure provides for a beverage comprising the aqueous emulsion, as described herein, wherein the beverage has a pH ranging from about 2 to about 4. According to certain embodiments, the beverages of the present disclosure may contain water and omega-3 fatty acids that are stable, bioavailable and without the fishy odor that is associated with the fish oils that contain the omega-3 fatty acids. For example, certain embodiments of the beverages may have a head space above the beverage that has a concentration of volatile amine compounds, such as TMA, that is less than the detection limit of the human nose. The polyunsaturated fatty acids, including omega-3 fatty acids, may come from a variety of sources including vegetable oil, fish oil, or an enriched blend of triglycerides as described herein. The beverage comprising the aqueous emulsions described herein may comprise an emulsifier as set forth herein. Other emulsifiers and other optional additives suitable for use in the emulsions and beverages of this disclosure are described in U.S. Pat. Nos. 6,126,980, issued to Smith et al., on Oct. 3, 2000; 5,431,940, issued to Calderas et al., on Jul. 11, 1995; and 6,326,040 B1, issued to Kearney et al., on Dec. 4, 2001.

In certain embodiments of the present disclosure, the beverages may have about 300 mg of omega-3 fatty acids per each serving, for example a serving from about 50 mL to about 500 mL. As used herein a “serving” may be as about 250 mL of prepared beverage, however, other serving sizes of the beverage are also contemplated, such as a serving size ranging from about 50 mL to about 1000 mL. Thus, a “serving” for a powder mix is about 250 mL of liquid after the powder is re-hydrated according to the powder's instructions. In other embodiments, the beverages may comprise at least about 150 mg of DHA and 150 mg of EPA in each serving. In other embodiments, the beverage may comprise at least about 250 mg of DHA and 250 mg of EPA in each serving and in still other embodiments the beverage may comprise at least about 350 mg of DHA and 350 mg of EPA in each serving. Other ratios of EPA and DHA in each serving of the beverage may include ratios as described in greater detail herein.

According to the various embodiments, the final beverage product has no perceptible fishy odor or fishy flavor. In certain embodiments, control of pH and/or redox potential of the emulsions, food products, or beverages are important methods for reducing or eliminating the fishy odor and fishy taste and oxidation products in the present compositions, thereby producing stabile compositions. Control of the redox potential of the emulsion and compositions comprising the emulsion is discussed in greater detail herein. For example, reducing the redox potential to a value less than 600 mV, and in other embodiments to redox values as recited herein, may result in reduced oxidation of the polyunsaturated fatty acids. Regulation of the pH of the emulsion and compositions comprising the emulsions is discussed in detail herein. According to certain embodiments, the beverages may have a pH of less than about 4.5. In certain embodiments, the beverages may have a pH ranging from about 4.0 to about 2.0, and in other embodiments ranging from about 3.7 to about 2.5. The beverages have no perceptible fishy odor and/or fishy or off flavor since the resulting beverages will have concentrations of volatile amine compounds, such as TMA, similar to those of the aqueous emulsions that are used to produce the beverage.

Natural Oils

Table 1 illustrates the compositions of certain commercially available fish oils and other natural oils rich in omega-3 fatty acids and suitable for use in the compositions and methods according to various embodiments of the present disclosure. The composition and methods of the present disclosure are not intended to be limited to any specific commercial fish oil and may incorporate or use other sources of fish oils, vegetable oils, concentrated or processed oils, and other esters of polyunsaturated fatty acids. Adding these oils to water to form the emulsions as described herein, without further treatment or additives will generally result in an emulsion or composition with a fishy odor and fishy or off flavor, due at least in part to the presence of residual amounts of volatile amines, such as trimethylamine, and other fatty acid oxidation products. Such an emulsion is generally not suitable to be used as a beverage, food product, or as a food or beverage additive since the fishy odor and/or fishy or off taste is considered unpleasant and is not acceptable to many consumers. As such, emulsions according to various embodiments of the present disclosure which can contain blends of esters, such as the fish oils disclosed herein, and have no perceptible smell or off-flavors, present a substantial improvement over the art.

TABLE 1
Examples of Commercially Available Oils Containing Omega-3 Fatty Acids

| Company | Sample | % omega-3 | % DHA | % EPA | % ALA | Source |
|---|---|---|---|---|---|---|
| Blue Pacific | Omega-3 | 10% = 2.2-2.5% | 0.7-1.2% | 1.2-1.8% | | Menhaden |
| | Antioxidant Blend | 20% = 4.4-5.0% | 1.1-2.4% | 2.4-3.6% | | fish oil |
| | | 30% = 6.6-7.5% | 2.1-3.6% | 3.6-5.4% | | |
| BASF | Dry n-3 18:12 | 35% | 12% | 18% | | fish |
| | Dry n-3 5:25C | 35% | 25% | 5% | | |
| Omega Pure | Omega Pure | 4.8% | | | | fish |
| | Lipsome-20 | | | | | |
| | Omega dry 1510 | 6% | | | 13% | |
| | Refined oil | 20-26% | 7-18% | 8-18% | | |
| Martex | DHASCO-S | | 38.3% | 1.44% | | algae |
| Pizzey's Milling | BevGrad | 30% | | | 22% | flaxseed |
| | Flaxseed | | | | | |
| DSM(roche) | Ropufa Oil | 30% | | | | |
| | Ropufa powder | | | | | |
| Loders | Marinol | Powder 18% | 160 mg/g | 20 mg/g | | fish |
| Croklaan | | Oil 45% | 15% | 21% | | |
| B.V.(lipid | | | | | | |
| nutrition) | | | | | | |
| Bioral | | 6-10% ropufa | | | | fish |
| Polar Foods, | HiOmega3 | 70.2% | | | 70.3% | Flax oil |
| Inc | RegRefin O-3 | 58.2% | | | 58.2% | |

Oil Processing

While the present disclosure is explained and exemplified with a blend of esters comprising polyunsaturated fats, concentrates of polyunsaturated fats, such as concentrates made from natural oils (such as marine oils and vegetable oils) may be utilized as the blend of esters. Various commercial processes may be used to produce concentrates suitable for use in the compositions and methods described herein. According to one embodiment, the process as described herein for producing a concentrate high in omega-3 fatty acids from commercially available natural oils may be used. The processing of fish oils, such as using the process as set forth herein, produces blends concentrated in omega-3 fatty acids, however, those skilled in the art will appreciate that the processes described herein can be used with any oil containing PUFAs. Fish oil, however, is an abundant source of PUFAs and omega-3 fatty acids, and may present additional challenges of possessing an undesired odors, undesired tastes, and off colors which may be addressed by the methods disclosed herein.

Fish oils suitable for use as the starting materials in the present disclosure include not only the fat and oils obtained from fish such as described herein, including sardine, pilchard, chub mackerel, pacific saury, salmon, trout, tuna and the like, expressed according to a conventional method, but also fats and oils removed from viscera of pollack, shark, etc. and also from such Mollusca as squid and/or cuttle fish, octopus, etc.

The fish oil used as the starting material in the present disclosure may be crude fish oil expressed from fish, but in certain embodiments to improve the efficiency of deodorization and molecular distillation in the later stages of processing, it may be desirable to subject the crude fish oil to acid refinement by means of phosphoric acid, sulfuric acid and the like, or to alkali treatment by means of caustic alkali and then further to treatment by preliminary refinement processes, such as deacidification, decoloration, dewaxing, etc., to obtain a product having higher content of triglycerides.

The crude fish oil may be pre-treated to clean and purify it. Those skilled in the art will appreciate that the amount and necessity of pre-processing will depend on the quality of the crude oil stream. Standard cleaning steps known to skilled artisans may be used alone or in combination with other methods. For example, filtration, adsorption, evaporation and steam stripping are all methods that may be used to clean the oil depending on the purity of the crude fish oil. In certain cases, the fish oil stream may be optionally filtered to remove solids. The filtered oil may then be fed into an adsorption column to remove certain color and odor bodies. Moreover, the fish oil stream may be optionally treated with steam to strip out certain free fatty acids and other volatile compounds.

After pre-treating, the fish oil is converted to esters of fatty acids via a transesterification reaction. The fish oil is treated with an excess of a lower molecular weight alcohol, such as, for example, methanol or ethanol, in the presence of a catalyst. Any of a variety of common catalysts can be used, such as basic catalysts, for example, sodium methoxide or potassium carbonate. Glycerol is the major by-product of the reaction of triglyceride oils with lower alcohols. Most of the glycerol is removed and the esterified oil is then moved to a different vessel.

After the glycerol is removed, the esterified fatty acids may optionally be flashed to remove excess of the lower molecular weight alcohol, which can be recycled if desired. After the optional flashing, the esterified fatty acids may be washed with water to remove water soluble bodies, such as, for example, any remaining glycerol, soap, lower alcohols, odor bodies, color bodies, and/or flavor bodies. The washed, esterified fatty acids are then dried to remove residual water and residual water soluble bodies.

Next, fractional distillation of the esterified fatty acids removes lower chain length esters and other residual low boilers, for example, certain lower alkyl esters. It is preferred that the C18 and lower esters are removed during distillation to increase the concentration of C20, C22 and longer esterified fatty acids, such as omega-3 fatty acids. While C18, α-linolenic acid is generally considered one of the beneficial omega-3 fatty acids, in certain embodiments, it is may be removed by distillation in order to increase the concentration of the longer C20 and C22 fatty acid esters, such as esters of EPA and DHA, respectively. By targeting the distillation process to extract the longer esterified fatty acids, some of the C18 α-linolenic acid will, of course, be retained, while the overall concentration of omega-3 fatty acids, and specifically, EPA and DHA, will be maximized in the final product.

Optionally, after distillation, a wiped film evaporator may be used to further purify the longer chain ester stream by removing some of the high boiling residual compounds. Having removed both high boiling residuals and low boiling residuals, the middle cut of purified and concentrated esters is then moved to a reaction vessel. Excess glycerol is added to this reactor in the presence of a catalyst, for example, a carbonate salt or sodium methoxide to reconvert the fatty acid esters to triglycerides by a second transesterification reaction. In certain embodiments, the concentrated product comprises a blend of triglycerides wherein at least about 50% of the fatty acid residues have a chain length of 20 carbons or more. In other embodiments, the product may comprise a blend of triglycerides wherein at least 60% of the fatty acid residues have a chain length of 20 carbons or more, in still other embodiments at least 70%, and in still other embodiments at least 80% by weight. In still other embodiments, the desired product has about 90%, by weight, triglycerides having high concentration of long chain polyunsaturated fatty acid residues, including long chain omega-3 fatty acid residues. The maximum desired concentration of monoglycerides is about 5% or less, and the maximum desired concentration for diglycerides is about 10% or less.

After the product stream has been converted to triglycerides, the product is purified and further concentrated. It is preferred that the product stream be treated to a water wash, which may be conducted in two stages. Potassium citrate may be added to convert the catalyst to a more easily removed compound, for example, potassium carbonate. In addition, any free fatty acids are converted to soap which are then removed with a water wash. After the water wash, the undesired components may be discarded or recycled and the triglyceride product stream is dried using a conventional dryer.

Following the removal of moisture, the concentrated triglyceride product stream may be bleached. Bleaching may be carried out in a column with silica gel, bleaching earth, alumina or the like. Certain color bodies, flavor bodies, odor bodies, and oxidized species may be removed by bleaching. The triglyceride product is then moved to an evaporation column where residual mono-esters and any remaining free fatty acids are removed. Optional steam stripping removes residual amounts of the color bodies, flavor bodies, odor bodies, peroxide, if present, and any residual free fatty acids. The cleaning and stripping steps described above may be used individually or in combination to achieve the desired purity for the final product.

At this stage the triglyceride mixture having a high concentration of omega-3 fatty acid esters should be substantially free of odor bodies and have no off flavors. As used herein, the term “substantially free of odor bodies” means that the triglyceride mixture has less than about 50 ppb trimethyl amine. The triglyceride mixture may be used in the compositions and methods described herein.

Various embodiments of specific methods for producing the concentrated omega-3 fatty acids are set for in greater detail in U.S. Provisional Application Ser. No. 60/815,991 filed Jun. 23, 2006, and the U.S. Non-Provisional Application entitled “Concentrated and Odorless Omega-3 Fatty Acids”, which claims priority to the above referenced provisional application and is filed on the same date as the present disclosure.

Nutrients

The aqueous emulsions of the present disclosure may optionally comprise nutrients, such as, vitamins and minerals, for example, but not limited to, calcium, iodine, sodium, potassium, vitamin C, vitamin E, vitamin A, niacin, thiamin, vitamin B₆, vitamin B₂, vitamin B₁₂, folic acid, selenium, pantothenic acid, and mixtures of any thereof. A preferred source of calcium may be a calcium citrate malate composition as described in U.S. Pat. Nos. 4,789,510; 4,786,518; and 4,822,847. Calcium in the form of calcium phosphate, calcium carbonate, calcium oxide, and calcium hydroxide in micron-sized particles having a dispersed particle size of about 100 nanometers (nm) or less, and in certain compositions, about 80 nm or less, may also be used. Additional calcium sources suitable for use herein include, for example, calcium citrate, calcium lactate, calcium citrate malate, and amino acid chelated calcium. The USRDI for calcium may range from 360 mg per 6 kg for infants to 1200 mg per 54-58 kg female, depending somewhat on age.

Commercial sources of iodine include iodine containing salts, e.g., sodium iodide, potassium iodide, potassium iodate, sodium iodate, or mixtures, preferably encapsulated potassium iodine of any thereof may also be used. In certain cases, the iodine salts may be encapsulated. Iodine may be added up to an amount equal to the current USRDI for iodine of 150 μg per serving.

Current USRDI values for various vitamins for healthy adults include: vitamin C (ascorbic acid) (60 mg), vitamin A as retinol (1 mg) or as β-carotene (3 mg), vitamin B₂ (1.7 mg), niacin (20 mg), thiamin (1.5 mg), vitamin B₆ (2.0 mg), folic acid (0.4 mg), vitamin B₁₂ (6 μg), and vitamin E (30 international units). The emulsions of the present disclosure may include various vitamins, as described herein, up to concentrations substantially equal to the USRDI amounts. Nutritionally supplemental amounts of other vitamins for incorporation into the certain embodiments of the emulsions described herein include, but are not limited to, vitamins B₆ and B₁₂, folic acid, niacin, pantothenic acid, folic acid, and vitamins D and E.

Moreover, the emulsions according to the various embodiments disclosed herein may also contain nutraceuticals such as glucosamine, phytosterols, chondroitin, soy isoflavones, and phytochemicals, for example bioactives obtained from botanical extracts, such as green tea, grape seeds, curry, ginger, broccoli, raspberry, and the like.

Coloring Agent

Small amounts of coloring agent, such as the FD&C dyes (e.g. yellow #5, blue #2, red # 40) and/or FD&C Lakes may be optionally added to the emulsions and/or products comprising the emulsions. Such coloring agents may be added to the emulsions for aesthetic reasons only. Preferred Lake dyes that can be used in certain embodiments of the present disclosure are the FDA approved Lake dyes, such as Lake red #40, yellow #6, blue #1, and the like. Additionally, a mixture of FD&C dyes and/or a FD&C lake dye in combination with other conventional food and food colorants can be used. The exact amount of coloring agent used will vary, depending on the agents used and the intensity desired in the finished product. The amount can be readily determined by one skilled in the art. Generally the coloring agent should be present at a level of from about 0.0001% to about 0.5%, preferably from about 0.004% to about 0.1% by weight of the emulsion or product comprising the emulsion. For example, when the emulsion is used in a beverage that is lemon flavored or yellow in color, riboflavin can be used as the coloring agent. For orange flavored beverages, P-carotene and riboflavin may both contribute to color the beverage orange.

Flavoring Agent

According to certain embodiments, the emulsions and compositions comprising the emulsions of the present disclosure may optionally comprise a flavoring agent consisting of any natural or synthetically prepared fruit or botanical flavorant or flavoring agent or mixtures of botanical flavorant and fruit juice blends. Such flavoring agents are added to the emulsions for aesthetic reasons only, and are not required to mask any fishy odor or off flavor or taste. Suitable natural or artificial fruit flavoring agents include, but are not limited to, lemon, orange, grapefruit, strawberry, banana, pear, kiwi, grape, apple, lemon, mango, pineapple, passion fruit, raspberry, and mixtures of any thereof. Suitable botanical flavors include, but are not limited to, jamaica, marigold, chrysanthemum, tea, chamomile, ginger, valerian, yohimbe, hops, eriodictyon, ginseng, bilberry, rice, red wine, mango, peony, lemon balm, nut gall, oak chip, lavender, walnut, gentiam, luo han guo, cinnamon, angelica, aloe, agrimony, yarrow, and mixtures of any thereof. For example, in one embodiment the flavoring agent may be added in from about 0.01% to about 10% by weight of the emulsion and in another embodiment from about 0.02% to 8% by weight of these flavors can be used. In other embodiments, dried fruit juices may also be used as flavoring agents. The actual amount of flavoring agent will vary and will depend on the type of flavoring agent used and the amount of flavor desired in the finished beverage. Other flavor enhancers, as well as flavorants such as chocolate, vanilla, etc., may also be used.

Sweetener

According to certain embodiments, the emulsions and compositions comprising the emulsions of the present invention may optionally comprise a sweetener or sweetening agent. Such sweetening agents may be added to the emulsions for aesthetic reasons only and are not required to mask any fish odor or off flavor or taste. Suitable particulate sugars can be granulated or powdered, and may include sucrose, fructose, dextrose, maltose, lactose, polyols, and mixtures of any thereof. In one particular embodiment, the sweetener may be sucrose. In other embodiments, artificial sweeteners may be utilized in the emulsions. Often gums, pectins and other thickeners may be used with artificial sweeteners, for example, to act as bulking agents and provide texture to the reconstituted dried emulsion. Various mixtures of sugars and artificial sweeteners may also be used.

In addition to the particulate sugars described herein, other natural or artificial sweeteners may also be incorporated therein. Suitable artificial sweeteners include, for example, saccharin, cyclamates, sucralose, acesulfam-K, L-aspartyl-L-phenylalanine lower alkyl ester sweeteners (e.g. aspartame), L-aspartyl-D-alanine amides as disclosed in U.S. Pat. No. 4,411,925 to Brennan et al., L-aspartyl-D-serine amides as disclosed in U.S. Pat. No. 4,399,163 to Brennan et al., L-aspartyl-L-1-hydroxymethylalkaneamide sweeteners as disclosed in U.S. Pat. No. 4,338,346 to Brand, L-aspartyl-1-hydroxyethyalkaneamide sweeteners as disclosed in U.S. Pat. No. 4,423,029 to Rizzi, L-aspartyl-D-phenylglycine ester and amide sweeteners as disclosed in European Patent Application 168,112 to J. M. Janusz, published Jan. 15, 1986, and the like. In one specific embodiment the artificial sweetener may be aspartame.

Preparation of the Emulsion

The emulsions of the present disclosure may be prepared from a variety of water sources, including, for example, deionized water, softened water, water treated by commercially available reverse osmosis processes, and distilled water.

Water may have high amounts of oxygen dissolved therein, resulting in a high redox potential for the water. Certain embodiments of the present disclosure may include treating the water to remove at least a portion of the dissolved oxygen. According to one embodiment, the process includes deoxygenating the water to reduce the concentration of oxygen in the water, or to eliminate all dissolved oxygen. According to one method of deoxygenation, the water is stripped of oxygen (and other dissolved gases) by bubbling carbon dioxide or other inert gas, such as nitrogen gas, through the water. The dissolved oxygen concentration in the water may also be reduced by heating the water to high temperatures, at which the solubility of the oxygen is reduced. According to certain embodiments, the oxygen level in the source water may be reduced to less than 5 parts per million (“ppm”), in other embodiments less than 3 ppm, and in still other embodiments less than 1 ppm.

The deoxygenation process may also remove other redox potential increasing agents, such as halide gases, for example chlorine gas, as well as volatile organic materials. Additionally, the water may be treated to have a minimal amount of the other electron acceptors that have redox potential greater than the PUFAs, including, for example, ozone, chlorides and hypochlorites, nitrates and nitrites, and metal ions of certain transition metals, such as ions of iron, copper, cobalt, nickel, and manganese.

The blend of esters comprising esters of polyunsaturated fatty acids, such as a fish oil or enhanced triglyceride blend, is admixed at the desired level, typically under stirring in a high shear mixer, followed by homogenization in an emulsifier. Typically, the admixing step is conducted under an inert gas blanket to exclude outside air and oxygen from the product. Finally, the emulsion may be packaged into glass or plastic bottles, or other suitable container. The plastic material of the bottle may be an oxygen-impermeable barrier and the bottle may be flushed with inert gas, such as nitrogen, prior to filling. Such oxygen-impermeable bottles are commercially available and will be known to those skilled in the art. In other embodiments, the emulsion may be added directly to a food product or a beverage.

The following representative examples are included for purposes of illustration and not limitation.

### EXAMPLES

### Example 1

In this Example, liquid beverage composition according to the present disclosure having the composition set forth in Table 2 is prepared.

TABLE 2
Composition of Beverage

| | % w/w |
|---|---|
| INGREDIENTS | |
| Water | 94.927 |
| Phosphoric Acid | 0.14 |
| Malic Acid | 0.22 |
| Whey Protein | 1.33 |
| Potassium citrate | 0.1 |
| Dextrose mono hydrate | 1.75 |
| Sucrolose | 0.02 |
| Sodium EDTA | 0.01 |
| FD&C Yellow # 6 | 0.0012 |
| FD&C Yellow # 5 | 0.0028 |
| Mango Flavor | 0.16 |
| Ascorbic acid | 0.024 |
| Lecithin | 0.015 |
| Ascorbyl palmitate | 0.02 |
| Concentrated Fish Oil (Twin Rivers Tech. Ohio) | 0.65 |
| D-Glucosamine hydrochloride | 0.63 |
| Total | 100 |
| EPA gm/250 mL | 0.5 |
| DHA gm/250 mL | 0.3 |
| EPA + DHA g/250 mL | 0.8 |
| Omega 3 FA | 1.0 |
| Conc. Fish Oil specification (Twin Rivers Tech. Ohio) | |
| EPA | 31.0 |
| DHA | 19.5 |
| Omega FA | 61.0 |

All the water soluble ingredients except for ascorbic acid are dissolved in the given amount of water. Whey protein is added through liquefier and blended to give clear solution. Ascorbic acid is added and dissolved. Separately, ascrobyl palmitate and lecithin are dissolved in fish oil by warming. The oil is then blended with beverage pre-mix by high shear mixer. The beverage is immediately emulsified at 3300 psi pressure, ascetically processed and packed. The resulting emulsion has a pH of 3.34, a specific gravity of 1.018 g/cc, and is physically stable, with no oil or cream separation. The beverage has no off odor or fishy odor.

### Example 2

In this Example, a liquid beverage composition according to the present disclosure having the composition set forth in Table 3 is prepared.

TABLE 3
Composition of Beverage

| | % w/w |
|---|---|
| INGREDIENTS | |
| Water | 57.31 |
| Orange Juice | 25.0 |
| Citric Acid | 0.37 |
| Sodium Citrate di hydrate | 0.17 |
| Sodium EDTA | 0.005 |
| Ascorbic Acid | 0.08 |
| Acyl Gellan | 0.032 |
| High Fructose Corn Syrup | 15.5 |
| FD&C Yellow # 6 | 0.0014 |
| Orange flavor | 0.1 |
| Lecithin | 0.015 |
| Alpha-tocopherol | 0.02 |
| Ropufa ‘30’ Fish Oil (Roche) | 1.3 |
| Phytosterol | 0.10 |
| Total | 100 |
| EPA gm/250 mL | 0.4 |
| DHA gm/250 mL | 0.3 |
| EPA + DHA g/250 mL | 0.7 |
| Omega 3 FA | 1.0 |
| Ropufa ‘30’ Fish Oil (Roche) Specification | |
| EPA | 13.5 |
| DHA | 8.0 |
| Omega FA | 30.0 |

All the water soluble ingredients except for ascorbic acid are dissolved in the given amount of water. Whey protein is added through liquefier and blended to give clear solution. Ascorbic acid is added and dissolved. Separately, alpha-tocopherol and lecithin are uniformly dispersed in fish oil. The oil is then blended with beverage pre-mix by high shear mixer. The beverage is immediately emulsified by at 3300 psi pressure, ascetically processed and packed. The resulting emulsion has a pH of 3.25, a specific gravity of 1.031 g/cc, and is physically stable, with no oil or cream separation. The beverage has no off odor or fishy odor.

### Example 3

In this Example, a powder beverage composition according to the present disclosure was prepared. The powder can be reconstituted with water to for a drinkable beverage. All the dry ingredients are mixed to get uniform blend. The composition of the powder composition is presented in Table 4.

TABLE 4
Composition of Beverage Powder

| | % w/w |
|---|---|
| INGREDIENTS | |
| Granulated Sugar | 71.6 |
| Citric Acid | 4.18 |
| Orange powder flavor | 1.57 |
| Sodium citrate | 2.19 |
| FD&C # 6 Al. Lake | 0.08 |
| FD&C # 5 | 0.02 |
| Xanthan Gum | 0.14 |
| FD&C Yellow # 6 | 0.00 |
| FD&C Yellow # 5 | 0.00 |
| Ascorbic acid | 0.68 |
| Ropufa‘10’ Powder (Roche) | 16.80 |
| Tricalcium phosphate | 1.50 |
| Aspartame | 1.23 |
| Niacinamide | 0.01 |
| Total | 100.00 |
| Omega 3 FA mg/250 mL | 390 |
| Ropufa ‘10’ Fish Oil (Roche) powder Specification | |
| Omega-3 FA | NLT 9.0% |

A ready-to-drink orange colored opaque beverage is prepared by dispersing 45 g of the powder in 1000 mL of water. The resulting beverage has a pH of 3.21. The resulting beverage contains 390 mg of omega-3 fatty acids per 250 mL serving. The beverage has no fishy odor.

### Example 4

In this Example, a concentrated fish oil emulsion composition according to the present disclosure is prepared. The concentrate emulsion can be diluted with water or other diluent to form a suitable beverage composition, for example, to form a drinkable beverage with desirable amounts of omega-3 fatty acids. The concentrated emulsion may also be used as a food additive by adding to a composition to form a food product. The composition of the emulsion is presented in Table 5.

All the water soluble ingredients except for ascorbic acid and erythorbic acid are dissolved in the given amount of water. Whey protein is added through liquefier and blended to give a clear solution. Ascorbic and erythorbic acids are added and dissolved. Separately, alpha-tocopherol, ascorbyl palmitate, and lecithin are uniformly dispersed in fish oil and warmed to dissolve the ascorbyl palmitate. The oil is then blended with the concentrate pre-mix by high shear mixer. The concentrate is immediately emulsified by at 3300 psi pressure, ascetically processed and packed. The resulting concentrated emulsion has a pH of 2.69 and is physically stable, with no oil or cream separation. The emulsion has no off odor or fishy odor.

TABLE 5
Concentrated Emulsion Composition

| | % w/w |
|---|---|
| INGREDIENTS | |
| Phosphoric acid | 0.7 |
| Malic acid | 1.1 |
| Whey protein | 0.75 |
| Potassium citrate | 0.5 |
| Sodium EDTA | 0.05 |
| Lecithin | 0.08 |
| Ascorbic acid | 0.13 |
| Erythorbic acid | 0.25 |
| Ropufa ‘30’ Fish Oil (Roche) | 8.25 |
| Ascorbyl palmitate | 0.1 |
| Alpha-tocopherol | 0.05 |
| Water | 85.04 |
| Ropufa ‘30’ Fish Oil (Roche) Specification | |
| EPA | 13.5 |
| DHA | 8.0 |
| Omega FA | 30.0 |
| Fish oil Concentrate emulsion contains | |
| EPA | 1.1 |
| DHA | 0.7 |
| Omega FA | 2.5 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous emulsion comprising:
water;
a blend of esters comprising esters of polyunsaturated fatty acids; and
one or more compounds capable of reducing a redox potential of the aqueous emulsion.

2. The aqueous emulsion of claim 1, wherein the esters of polyunsaturated fatty acids are esters of omega-3 fatty acids.

3. The aqueous emulsion of claim 2, wherein the esters of omega-3 fatty acids are triglycerides having at least one omega-3 fatty acid ester.

4. The aqueous emulsion of claim 1, wherein the one or more compounds capable of reducing the redox potential of the aqueous emulsion are selected from the group consisting of a compound that lowers the pH of the aqueous emulsion, a redox modulator, a reducing agent, a chelator, an antioxidant, and combinations of any thereof.

5. The aqueous emulsion of claim 1, wherein the one or more compounds capable of reducing the redox potential of the aqueous emulsion is a redox modulator selected from the group consisting of ascorbic acid, ascorbyl palmitate, sodium bisulfite, erythorbic acid, sulfhydryl containing amino acid residues, polyphenols, flavinoids, soluble dietary fibers, and combinations of any thereof.

6. The aqueous emulsion of claim 5, wherein the redox modulator is one of ascorbic acid, erythorbic acid, and combinations thereof.

7. The aqueous emulsion of claim 1, wherein the one or more compounds capable of reducing the redox potential of the aqueous emulsion is a chelator selected from the group consisting of ethylenediamine tetraacetic acid, citric acid, tartaric acid, ascorbic acid, polyphosphates, esters or salts thereof, and combinations of any thereof.

8. The aqueous emulsion of claim 7, wherein the chelator is capable of binding to a metal ion selected from the group consisting of Fe²⁺, Cu²⁺, Mn²⁺, Co²⁺, Fe³⁺, Mn, Ni, and mixtures of any thereof.

9. The aqueous emulsion of claim 1, wherein the one or more compounds capable of reducing the redox potential of the aqueous emulsion is a combination of a water soluble antioxidant and an oil soluble antioxidant.

10. The aqueous emulsion of claim 9, wherein the water soluble antioxidant is selected from the group consisting of ascorbic acid, erythorbic acid, a botanical extract, rosemary extract, green tea extract, and combinations thereof.

11. The aqueous emulsion of claim 9, wherein the oil soluble antioxidant is selected from the group consisting of vitamin E, tocopherols, ascorbyl palmitate, butylated hydroxyanisole, butylated hydroxytoluene, and combinations of any thereof.

12. A method of reducing oxidation in an aqueous emulsion comprising:
adding a compound to the aqueous emulsion,
wherein the aqueous emulsion comprises water and a blend of esters comprising esters of polyunsaturated fatty acids, and the compound is capable of reducing a redox potential of the aqueous emulsion.

13. The method of claim 12, wherein the esters of polyunsaturated fatty acids comprise esters of omega-3 fatty acids.

14. The method of claim 13, wherein the esters of omega-3 fatty acids are triglycerides having at least one omega-3 fatty acid ester

15. The method of claim 12, wherein the blend of esters is a fish oil.

16. The method of claim 12, wherein the compound is selected from the group consisting of a compound that lowers the pH of the aqueous emulsion, a redox modulator, a reducing agent, a chelator, an antioxidant, and combinations of any thereof.

17. A method of improving the stability of an aqueous emulsion comprising omega-3 fatty acid esters comprising:
adding a compound to the aqueous emulsion, wherein the compound is capable of reducing a redox potential of the aqueous emulsion.

18. The method of claim 17, wherein the compound is selected from the group consisting of a compound that lowers the pH of the aqueous emulsion, a redox modulator, a reducing agent, a chelator, an antioxidant, and combinations of any thereof.

19. A method of improving the stability of an aqueous emulsion comprising omega-3 fatty acid esters, the method comprising:
reducing a redox potential of the aqueous emulsion to less than 600 mV.

20. The method of claim 19, wherein reducing the redox potential of the aqueous emulsion comprises:
adding a compound selected from the group consisting of a compound that lowers the pH of the aqueous emulsion, a redox modulator, a reducing agent, a chelator, an antioxidant, and combinations of any thereof, to the aqueous emulsion.
